(19) United States
(12) Patent Application Publication
(10) Pub. No.: US 2004/0072369 A1
(43) Pub. Date: Apr. 15, 2004

(54) Methods for assaying for urea and kits for use therein

(75) Inventors: Harry L. T. Mobley, Parkville, MD (US); Richard B. Thompson, Baltimore, MD (US); Jonathan D. Dattelbaum, Baltimore, MD (US)

Correspondence Address: SUGHRUE MION, PLLC, 2100 Pennsylvania Avenue, NW, Washington, DC, 20037-3213, US

(73) Assignee: UNIVERSITY OF MARYLAND, BALTIMORE

(21) Appl. No.: 10/441,247

(22) Filed: May 20, 2003

Related U.S. Application Data
(60) Provisional application No. 60/381,946, filed on May 20, 2002.

(51) Int. Cl. G01N 33/543

(52) U.S. Cl. 436/518

(57) ABSTRACT

Methods for assaying for urea in a test sample using a polypeptide comprising UreR or a urea binding fragment thereof, and fluorescence spectroscopy are disclosed, as well as a biosensor and kits for use in said methods.

### CROSS-REFERENCE TO RELATED APPLICATION

This application claims benefit of Provisional Application No. 60/381,946, filed May 20, 2002; the disclosure of which is incorporated herein by reference.

This work was supported by Public Health Service grant AI23328 and by the National Institutes of Health. The United States Government has certain rights to this invention.

### FIELD OF THE INVENTION

The present invention relates to methods for assaying for urea in a test sample using a polypeptide comprising UreR or a urea binding fragment thereof and fluorescence spectroscopy, and to biosensors and kits for use in said methods.

### BACKGROUND OF THE INVENTION

Urinary tract infection (UTI) is one of the most commonly cited causes of hospital visits for kidney and urologic disease in the United States (Anonymous, “Ambulatory Care Visits to Physician Offices, Hospital Outpatient Departments, and Emergency Departments”, In C. NCHS, HHS (ed.), United States (1998)). Proteus mirabilis is one organism responsible for acute and chronic urinary tract infections, particularly in individuals with long-term indwelling catheters or structural abnormalities of the urinary tract (Warren et al, J. Infect. Dis., 146:719-723 (1982)). To colonize the urinary tract, P. mirabilis must utilize a still undetermined number of virulence factors including fimbriae (Bahrani et al, Infect. Immun., 62:3363-3371 (1994); Massad et al, Infect. Immun., 62:1989-1994 (1994a); and Massad et al, Infect. Immun., 62:536-542 (1994b)), hemolysin (Toth et al, Acta Microbiologica et Immunologica Hungarica, 47:457-470 (2000)), flagella (Mobley et al, Infect. Immun., 64:5332-5340 (1996)), immunoglobulin A-degrading metalloprotease (Senior et al, J. Med. Microbiol., 24:175-80 (1987); and Wassif et al, J. Bacteriol., 177:5790-5798 (1995)); and a urea-inducible urease (Jones et al, J. Bacteriol., 171:6414-6422 (1989)).

Urease, which catalyzes the hydrolysis of urea into ammonia and carbon dioxide, is a well-recognized virulence determinant of P. mirabilis. Local elevation in pH caused by urease activity initiates the precipitation of normally soluble calcium and magnesium salts in the form of bladder and kidney stones, which are hallmarks of P. mirabilis infection (Dumanski et al, Infect. Immun., 62:2998-3003 (1994); and Griffith et al, Invest. Urol., 13:346-350 (1976)). A urease-deficient mutant, in which ureC was insertionally inactivated, is significantly attenuated and causes less histological damage in the urinary tract of transurethrally infected CBA mice (Johnson et al, Infect. Immun., 61:2748-54 (1993); and Jones et al, Infect. Immun., 58:1120-1123 (1990)).

UreR, a member of the AraC/XylS family of transcriptional activators, promotes transcription of the genes encoding urease structural subunits and accessory proteins, ureDABCEFG, as well as its own transcription in the presence of urea (D'Orazio et al, J. Bacteriol., 175:3459-3467 (1993); and Nicholson et al, J. Bacteriol., 175:465-473 (1993)). It has been hypothesized that urea might directly bind to UreR, thereby causing a conformational change in protein structure that promotes stronger binding of UreR to specific operator sequences upstream of both ureR and ureD, thus recruiting RNA polymerase and activating transcription. The transcriptional activity and DNA binding characteristics of the E. coli plasmid-encoded UreR have been studied (D'Orazio et al, Mol. Microbiol., 16:145-155 (1995); D'Orazio et al, Mol. Microbiol., 21:643-655 (1996); and Thomas et al, Mol. Microbiol., 31:1417-1428 (1999)). From this work, three urea-dependent UreR-inducible promoters in the urease gene cluster preceding ureR, ureD, and ureG were identified and a 13-bp consensus DNA binding site was elucidated.

Functional roles have been assigned to specific structural domains of P. mirabilis UreR (Poore et al, J. Bacteriol., 183:4526-4535 (2001)). A translational fusion between the known leucine zipper dimerization domain (amino acids 302-350) of C/EBP and the C-terminal region of UreR (amino acids 164-293) activates transcription from the ureD promoter, thus localizing the DNA-binding domain to the C-terminus of UreR and demonstrating a requirement for dimerization of UreR monomers. To localize the UreR dimerization domain, a translational fusion between the UreR N-terminal domain (amino acids 1-182) and the DNA-binding domain of LexA (amino acids 1-87), which only binds to its operator site as a dimer, was expressed; the fusion protein was found to retard mobility of a DNA fragment containing this site, as determined by gel shift. These and other data support the hypothesis that the UreR dimerization domain resides in the N-terminal region.

While a link between urea, UreR, and transcriptional activation of urease operon has been clearly demonstrated, there have been insufficient data to show that UreR directly interacts with urea. In the present invention, it has been demonstrated for the first time that UreR interacts with urea by using fluorescence spectroscopy to detect conformational changes in UreR in response to interaction with urea.

There is a clear clinical need for determination of urea in blood (“blood urea nitrogen”, or BUN), as it represents one of the most commonly performed clinical tests; millions are performed in the U.S. every year by thousands of clinical laboratories. Ordinary BUN levels are in the range of 2.5 to 10.7 mM, but altered levels are observed in the presence of many serious diseases, including renal disease, urinary tract obstruction, leukemia, liver failure, and gastrointestinal bleeding (Merck Manual of Diagnosis and Therapy, 17th Edition, Table 296-4, p. 2547). Furthermore, patients undergoing renal dialysis and their physicians would benefit from a continuous measure of the progress of urea removal as an index of the progress of the dialysis procedure. Also, to better understand the biochemistry and metabolism of urea, and particularly its role in urolithiasis, gastric disease, and kidney failure, it is desirable to be able to measure urea. For such scientific purposes, it is desirable to be able to quantify urea continuously, and to image concentrations of urea inside and outside cells in the fluorescence microscope, both in vitro and in vivo. It is particularly desirable to have indwelling sensors in some of these applications to measure levels in situ. It is also desirable to be able to measure urea levels significantly below the ordinary clinical range cited above.

The standard test for BUN is calorimetric, and is adequately sensitive, specific, reliable, and inexpensive. However, the test determines the level in a discrete blood sample, and thus does not measure the level continuously, nor report the level in real time, nor respond rapidly and reversibly to changes in urea level. The calorimetric test is inadequately sensitive for use as a research tool, and does not permit imaging of urea levels in tissue, nor detection through optical fiber endoscopy. Other analytical tests are known to the art for urea, including electrochemical, calorimetric, and fluorescence based tests. The non-optical electrochemical tests do not permit urea levels to be imaged in tissues or other milieus. Some of the optical tests have been adapted for use with optical fibers. Fluorescence based analyses are desirable because they can be very sensitive (typically one thousand-fold more sensitive than calorimetric analyses) and permit imaging of analyte concentrations in the fluorescence microscope. Moreover, fluorescence-based analyses which transduce the analyte level as a change in fluorescence intensity ratio, anisotropy, or lifetime are especially desirable because they are freer from artifact, easier to calibrate, and much more accurate than simple fluorescence intensity measurements (Lakowicz, Priniciples of fluorescence spectroscopy, 2 ed., Plenum Press, New York (2000); Thompson et al, Anal. Chem., 70:4717-4723 (1998); and Thompson et al, Anal. Chem., 70:1749-1754 (1998)). However, most of the current optical tests use a consumable reagent that does not permit a reversible response and continuous monitoring of urea levels without a sampling device. Thus, a determination of urea that is rapidly reversible, capable of continuous monitoring in real time, sensitive, selective, and is transduced by a fluorescence ratiometric or lifetime change would be desirable both as a research tool and clinically.

### SUMMARY OF THE INVENTION

Accordingly, an object of the present invention is to provide a method for assaying for the presence of urea in liquid samples and tissue.

An additional object of the present invention is to provide a method for assaying for the presence of urea in real time, rapidly and reversibly.

Still another object of the present invention is to provide a method for assaying for the presence of urea using an optical fiber, e.g., in optical fiber endoscopy.

Yet another object of the present invention is to provide reagents and a biosensor and kit for use in said method.

These and additional objects of the present invention, which will be apparent from the detailed description of the invention provided hereinafter have been met in one embodiment by a method for assaying for the presence of urea in a test sample comprising:

(A) contacting said test sample with a member selected from the group consisting of:

(1) a polypeptide comprising at least a urea binding fragment of UreR, wherein optionally, a tryptophan residue has been substituted for at least one amino acid residue therein,

(2) a polypeptide comprising at least a urea binding fragment of UreR covalently linked to a solvent-sensitive fluorophore,

(3) a polypeptide comprising at least a urea binding fragment of UreR in the presence of a fluorescent thiourea, and

(4) a fusion protein comprising a polypeptide comprising at least a urea binding fragment of UreR and a fluorescent protein;

(B) measuring

(1) a change in intrinsic fluorescence of tryptophan in said polypeptide,

(2) a change in fluorescence of said solvent-sensitive fluorophore,

(3) a change in fluorescence of said fluorescent thiourea, or

(4) a change in fluorescence of said fluorescent protein, respectively,

as a result of binding of urea present in said test sample to said polypeptide.

### BRIEF DESCRIPTION OF THE DRAWINGS

FIG. 1 shows UreR-GFP induced in P. mirabilis using a Western blot of induced cell lyzates and anti-GFP.

FIG. 2 shows Lys, Asp, Cys and His point mutants of UreR. Saturating alanine-scanning mutagenesis was used to create individual point mutants of all Asp, Cys, His and Lys residues in the N-terminal domain of P. mirabilis UreR (amino acids 1-184). Mutated amino acid residues are numbered.

FIG. 3 shows the induction of β-galactosidase activity from a P_{ureD}-lacZ reporter construct by UreR site-directed mutants. β-galactosidase activity was measured in E. coli Top10 co-transformed with the reporter construct and pBAD-UreR-MycHis₆ chimeric fusion vectors. Cultures were grown to mid-log phase and induced with 0.2% (w/v) arabinose in the presence or absence of 100 mM urea for 90 min. All constructs were assayed in duplicate in five independent experiments.

FIG. 4 shows the purification of nUreR single Trp mutants using Ni-NTA affinity chromatography. Representative Coomassie-stained 15% (w/v) SDS-PAGE of the nUreR single Trp mutants are shown. Proteins were eluted with 250 mM imidazole.

FIGS. 5A-5D show nUreR Trp mutant emission spectra as a function of urea concentration. Addition of increasing urea concentrations (0-150 mM) resulted in a decrease or no change in emission intensity for each mutant. Protein samples (2.0 μM) in 50 mM PBS (pH 7.0), were excited at 280 nm at room temperature. Note different scales for relative fluorescence intensity.

FIG. 6 shows binding of the Y54W nUreR mutant to urea. Three independent experiments were used to calculate the binding constant for this mutant. ΔTrp Emission is the difference in Trp emission intensity with and without urea. The binding constant was determined by fitting the data to the corresponding Hill parameters.

FIGS. 7A-7D show frequency-dependent phase and modulation fluorescence measurements of single Trp nUreR mutants (2.0 μM) in the absence (▪) and presence (●) of 100 mM urea, which are fit to apparent fluorescence lifetimes. Samples were excited at 295 nm to minimize any contaminating Tyr emission at room temperature.

FIG. 8 shows urea-sensitive changes in fluorescence of NBD-labeled nUreR-Cys59. Each point represents the peak NBD fluorescence signal at 535 nm in the absence or presence of 1.0 mM urea. Measurements were performed in quadruplicate.

FIG. 9 shows a ratiometric excitation FRET approach for a urea biosensor. Diethylaminocoumarin isothiourea (DACITU), a fluorophore, is the donor; and yellow fluorescent protein (YFP), is the acceptor.

FIG. 10 shows an oblique view of an apparatus for fluorescence anistropy sensing of urea in accordance with an aspect of the present invention.

FIG. 11 is a top view of the apparatus shown in FIG. 10.

### DETAILED DESCRIPTION OF THE INVENTION

As discussed above, in one embodiment, the present invention relates to a method for assaying for the presence of urea in a test sample comprising:

(A) contacting said test sample with a member selected from the group consisting of:

(1) a polypeptide comprising at least a urea binding fragment of UreR, wherein a tryptophan residue has been substituted for at least one amino acid residue in said fragment,

(2) a polypeptide comprising at least a urea binding fragment of UreR covalently linked to a solvent-sensitive fluorophore,

(3) a polypeptide comprising at least a urea binding fragment of UreR in the presence of a fluorescent thiourea, and

(4) a fusion protein comprising a polypeptide comprising at least a urea binding fragment of UreR and a fluorescent protein;

(B) measuring

(1) a change in intrinsic fluorescence of tryptophan in said polypeptide,

(2) a change in fluorescence of said solvent-sensitive fluorophore,

(3) a change in fluorescence of said fluorescent thiourea, or

(4) a change in fluorescence of said fluorescent protein, respectively,

as a result of binding of urea present in said test sample to said polypeptide.

The particular test sample employed is not critical to the present invention. For example, the test sample may be any tissue or liquid solution, such as serum, plasma, intracellular fluid, urine or water. Thus, the process of the present invention may be carried out, in vivo, in vitro or in situ.

The polypeptide employed in the present invention is not critical as long as it contains at least a urea binding fragment of UreR. In particular, the polypeptide preferably comprises amino acids 54-59 of UreR, preferably amino acids 20-80 of UreR, more preferably at least amino acids 1-184 of UreR.

The UreR may be Proteus mirabilis UreR (Accession No. CAA79243), Providencia stuartii UreR, E. coli UreR (Accession No. AAA24750), Corynebacterium glutamicum UreR (Accession No. AB029154), or Vibrio parahaemolyticus UreR (Park et al, Infect. Immun., 68:5742-5768 (2000); Accession No. AB038238).

The polypeptide may be naturally-occurring or recombinantly or synthetically produced. In addition, the polypeptide may be a fusion protein containing a tag, e.g., 6xHis, Flag, biotinylational signal, or Maltose binding protein, for ease of purification.

The polypeptide is preferably a polypeptide (amino acids 1-184 of UreR) obtained from Proteus mirabilis (nUreR), more preferably nUreR which has been mutated so as to substitute tyrosine at position 54 with tryptophan (Y54W nUreR).

The particular amount of the polypeptide employed is not critical to the present invention.

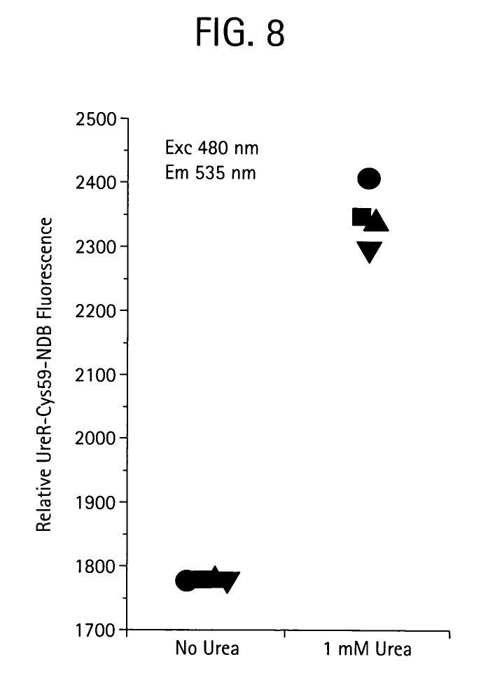

Analytes such as metal ions (Thompson, In: Lakowicz (ed.), Topics in Fluorescence Spectroscopy Vol. 2: Principles, pp.345-365, Plenum Press, New York (1991); Thompson et al, Biosensors Bioelectronics, 11:557-564 (1996); Thompson et al, Biophys J., 70:WP296-WP296 Part 2 (1996); Thompson et al, Analytical Chemistry, 65:730-734 (1993a); Thompson et al, Anal. Chem., 65:853-856 (1993b); Thompson et al, Anal. Biochem., 227:123-128 (1995a), Thompson et al, J. Fluorescence, 5:123-130 (1995b); Thompson et al, In: Cohn (ed.) SPIE Conference on Clinical Diagnostic Systems, pages 88-93, Society of Photooptical Instrumentation Engineers, San Jose, Calif. (2001); and Thompson et al J. Neurosci. Meth., 96:35-45 (2000)) and glutamine (Dattelbaum et al, Anal Biochem., 291:89-95 (2001)) can be determined by measuring fluorescence changes of a suitable fluorescent label conjugated to a suitable binding protein. Thus, the polypeptides of the present invention, which have been found in the present invention to serve as a high affinity, high selectivity binding protein for urea, can be modified by site-directed mutagenesis to permit conjugation of e.g., thiol-specific, solvent-sensitive fluorescent labels to cysteine residues inserted in the sequence. Binding of urea to this conjugate can causes changes in the fluorescence of a suitable solvent-sensitive fluorescent label attached at a particular position. In general the solvent-sensitive fluorophores respond best when in close proximity to the analyte binding site; this is likely due to the change in the ability of the protein and solvent structure to relax around the fluorescent label following excitation. An example of such response is given in FIG. 8 herein (Example 5), which shows urea-dependent changes in fluorescence intensity of NBD-variant.

Solvent-sensitive fluorophores, which are well-known in the art, are fluorophores whose fluorescence properties (most commonly, emission spectra or quantum yield) are sensitive to (i.e., change depending upon) certain characteristics of their surrounding milieu (“the solvent”), especially the polarity of the solvent and the presence of exchangeable hydrogen atoms (e.g., a “protic” solvent).

The particular solvent-sensitive fluorophore employed is not critical to the present invention. For example, the solvent-sensitive fluorophore may be:

4-chloro-7-nitrobenz-2-oxa-1,3-diazole (NBD-Cl),

5-((((2-iodoacetyl)amino)ethyl)amino) naphthalene-1-sulfonic acidio (IAEDANS),

7-fluorobenz-2-oxa-1,3-diazole-4-sulfonamide (ABD-F),

2-(4′-maleimidylanilino)naphthalene-6-sulfonic acid, sodium salt (MIANS), and

1-(2-maleimidylethyl)-4-(5-(4-methoxyphenyl)oxazol-2-yl) pyridinium methanesulfonate (pympo maleimide).

The solvent-sensitive fluorophore may be covalently linked to the polypeptide through the sulfhydryl of a cysteine substitution at position 59 of UreR, or through primary amines in the polypeptide sequence, e.g., using N-hydroxy succinimidyl fluorescent derivatives.

Preferably, the polypeptide is Proteus mirabilis nUreR, wherein the sulfhydryl of the cysteine at position 59 of the polypeptide is covalently linked to said solvent-sensitive fluorophore, and wherein said solvent-sensitive fluorophore is NBD iodacetamide.

Alternatively, a fluorescent thiourea may be used to compete with urea in solution for a binding site on the polypeptide; binding of the fluorescent thiourea results in an increase in its fluorescence anisotropy (polarization). In this case the protein is not fluorescent-labeled. If the urea concentration is high, a smaller fraction of the fluorescent thiourea is bound and a reduced anisotropy is observed; at lower urea concentrations, more of the thiourea is bound and the anisotropy is higher.

The particular fluorescent thiourea employed is not critical to the present invention. For example, the fluorescent thiourea may be fluorescein thiourea, tetramethylrhodamine thiourea or Eosin thiourea.

The particular amount of fluorescent thiourea employed is not critical to the present invention. Generally, the amount of fluorescent thiourea employed will be about 1.0 pM to 1.0 mM, preferably about 1.0 nM to 1.0 μM.

The particular ratio of polypeptide to fluorescent thiourea employed is not critical to the present invention.

Alternatively, a fusion protein can be created by fusing the genes for the polypeptide and those of a suitable fluorescent protein, such as the Green Fluorescent Protein from Aequorea (Miyawaki et al, Proc. Natl. Acad. Sci., USA, 96:2135-2140 (1999)). The optimum position of the fluorescent donor (fixed by its point of attachment to the polypeptide) for such energy transfer-based sensor transducers and its suitability may be calculated from Forster theory according to Thompson et al, J. Biomed. Opics, 1:131-137 (1996). In this case the acceptor is a thiourea made from the largely non-fluorescent dye Malachite Green isothiocyanate, which thiourea competes for the urea binding site. In the absence of urea a relatively large fraction of the fluorescent-labeled protein has Malachite green thiourea bound to its binding site, such that efficient resonant energy transfer occurs and the donor fluorescent label exhibits reduced intensity and lifetime, and increased anisotropy. In the presence of urea, fewer of the binding sites are occupied with the acceptor, energy transfer is almost absent, and the fluorescent label has unchanged intensity, lifetime, or anisotropy.

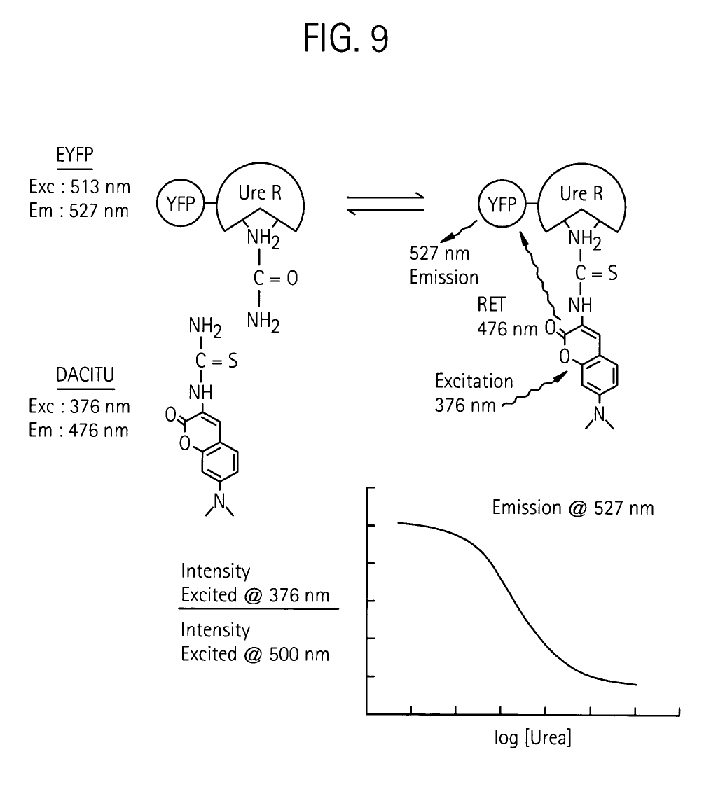

Alternatively, a fluorescent label on the protein can be used in combination with a fluorescent thiourea. In this case, the fluorescence spectra of the label and thiourea are chosen so that the two are capable of serving as acceptor and donor, respectively (see FIG. 9).

For example, where the label is Yellow Fluorescent Protein and the thiourea is diethylaminocoumarin isothiourea (DACITU), the concentration of urea is transduced as a change in the ratio of fluorescent intensity observed at 527 nm with excitation at 376 nm, to the intensity at the same emission wavelength excited at 513 nm. At low urea concentrations the UreR binding site is saturated with DACITU and the ratio of intensity excited at 376 nm to that excited at 513 nm is relatively high, because the energy from the bound DACITU is efficiently transferred to the YFP due to its close proximity. At higher urea concentrations less DACITU is bound, there is less energy transfer, and the ratio of intensity excited at 376 nm to that excited at 513 nm is reduced. The advantages of such intensity radiometric fluorescence determinations are well-known to the art (Tsien, Annu. Rev. Neurosci., 12:227-253 (1989); and Haugland, Molecular Probes' Handbook of Fluorescent Probes and Research Chemicals, 6^{th} ed., Eugene, Oreg. (1996)).

The particular fluorescent protein employed is not critical to the present invention, as long as the thiourea employed is fluorescent and can serve as a suitably efficient resonance energy transfer donor for the chosen fluorescent protein; suitability may be judged from the overlap integral and, thus, energy transfer efficiency, which is calculated by methods well-known to the art. For example, the fluorescent protein may be Aequorea Green Fluorescent protein, Yellow Fluorescent Protein, Blue Fluorescent Protein, or DsRed.

The particular amount of fusion protein employed is not critical to the present invention. Generally, the amount of fusion protein employed will be about 1.0 pM to 1.0 mM, preferably about 1.0 nM to 1.0 μM.

The change in fluorescence measured in the present invention is preferably at least a change in fluorescence intensity, a change in fluorescence anisotropy, a change in fluorescence polarization, a change in fluorescence emission or a change in excitation spectrum. The choice of wavelengths is determined from the spectral properties of the fluorophores (and any absorbents present as well) as is conventional in the art.

The availability of a system that exhibits changes in its fluorescence anisotropy with varying concentrations of urea allows one to make a biosensor for urea based on this binding reaction. This approach to biosensor design is based on UreR binding tightly to its target DNA in the presence of urea. In a simple embodiment, a DNA entrapping fluorescent-labeled oligonucleotide (target of binding) and UreR can be used at a suitable concentration within a urea-permeable chamber (such as a dialysis membrane) and fluorescence anisotropy (an index of the freedom of movement of labeled DNA in this case) can be measured through a transparent wall of the chamber, as the chamber is exposed to various concentrations of urea.

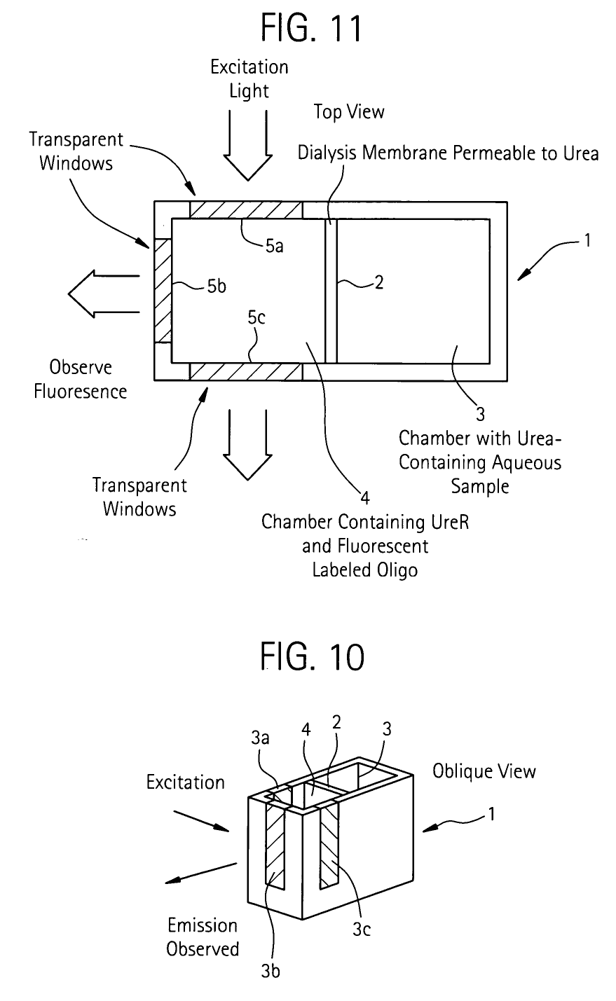

In one embodiment, as shown in FIGS. 10 and 11, the biosensor or apparatus for fluorescence anistropy of urea 1 includes a dialysis membrane 2 that is permeable to urea and that divides the apparatus 1 into a first chamber 3 and a second chamber 4. The second chamber 4 includes transparent windows 5a, 5b, and 5c, which permit the transmission of an excitation light and the observation of fluorescence anistropy. In practice, a urea-containing aqueous sample is present in the first chamber 3. A protein, such as UreR, and a target DNA, such as a fluorescent-labeled oligonucleotide comprising at least a UreR binding fragment of an UreR-inducible promoter oligonucleotide, are present in the second chamber 4. An excitation light is directed into the second chamber 4 through one of the transparent windows, e.g., transparent window 5a, and allowed to exit through another transparent window, e.g., opposing transparent window 5c. The fluorescence anistropy can then be observed through the third transparent window, e.g., transparent window 5b.

The advantages of the above approach are that it is reversible and non-destructive of the sample, that it can be carried out intracellularly by microinjecting the components, and that it is quite sensitive. Anisotropy-based biosensing of metal ions has previously been demonstrated (Thompson et al, Anal. Chem., 70:4717-4723 (1998a); Thompson et al, Anal. Chem., 70:1749-1754 (1998b); and Thompson, In: Lakowicz (ed.), Topics in Fluorescence Spectroscopy Vol. 2: Principles, pages 345-365, Plenum Press, New York (1991)).

While a UreR-DNA complex is formed in the absence of urea, the DNA binding affinity is greatly increased in the presence of urea. Using anisotropy measurements, it is possible to determine the percentages of free and bound DNA (LeTilly et al, Biochem., 32:7753-7758 (1993)). Short oligonucleotides corresponding to the target promoter that precede the ureD and ureR genes (pureD and pureR) can be synthesized and labeled at the 5′ end with fluorescent labeled DNA, e.g., fluorescein-isothiocyanate (FL-DNA). When oligos longer than 12 nucleotides are used, a longer lifetime fluorescent label, such as PyMPO, provides a better response. At constant protein and DNA concentrations, the anisotropy of the system can be measured as a function of increasing urea concentrations. The amount of the polypeptide and FL-DNA can be optimized to obtain the largest possible increase in anisotropy. Measurements can be carried out in a spectrofluorimeter fitted with thin film polarizers.

Thus, in another embodiment, the present invention relates to a method for assaying for urea in a test sample comprising:

(A) contacting a test sample with a polypeptide comprising UreR, and a fluorescent-labeled oligonucleotide comprising at least a UreR binding fragment of a UreR-inducible promoter, wherein said contacting is carried under conditions such that said UreR binds to said oligonucleotide; and

(B) measuring a change in anisotropy as a result of binding of urea present in said test sample to a complex formed between said oligonucleotide and said polypeptide.

The particular size of the oligonucleotide is not critical to the present invention as long as it is capable of binding to the DNA binding domain of UreR, which in the case of P. mirabilis UreR is amino acids 180-293.

Further, the particular UreR-inducible promoter to which the oligonucleotide corresponds is not critical to the present invention. For example, the UreR-inducible promoter may be pureD and pureE.

The particular amount of fluorescent-labeled oligonucleotide employed is not critical to the present invention, Generally, the amount employed will be in the range of 1.0 nM to 1.0 μM, preferably 100 μM.

Conditions under which UreR binds to the oligonucleotide are well-known in the art, and are not critical to the present invention.

In still another embodiment, the present invention relates to a kit for assaying for the presence of urea in a test sample comprising:

(A) a member selected from the group consisting of:

(1) a polypeptide comprising at least a urea binding fragment of UreR, wherein a tryptophan residue has been substituted for at least one amino acid residue in said fragment,

(2) a polypeptide comprising at least a urea binding fragment of UreR covalently linked to a solvent-sensitive fluorophore,

(3) a polypeptide comprising at least a urea binding fragment of UreR in the presence of a fluorescent thiourea, and

(4) a fusion protein comprising a polypeptide comprising at least a urea binding fragment of UreR and a fluorescent protein; and

(B) a member selected from the group consisting of a buffer solution, a sample diluent; a vessel for mixing said polypeptide and buffer solution or sample diluent, and a standard.

The following examples are provided for illustrative purposes only, and are in no way intended to limit the scope of the present invention.

### EXAMPLE 1

As discussed above, P. mirabilis, a cause of complicated UTI, expresses urease when exposed to urea. While it is recognized that the positive transcriptional activator UreR induces gene expression, the level of expression of the enzyme during experimental infection was not known prior to the present invention.

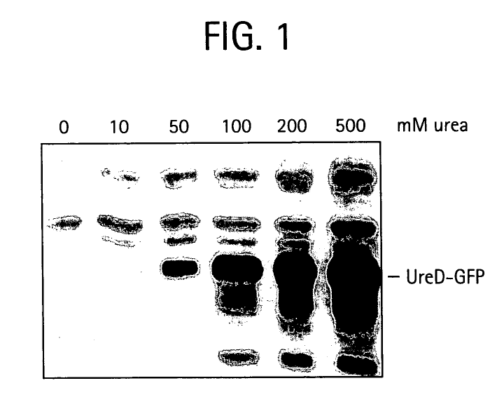

Thus, to investigate in vivo expression of P. mirabilis urease, the gene encoding green fluorescent protein (GFP) was used to construct reporter fusions. Translational fusions of urease accessory gene ureD, which is preceded by a urea-inducible promoter, were made with gfp (modified to express S65TIV68L/S72A, a well-known variant of gfp). The constructs were confirmed by sequencing of the fusion junctions. UreD-GFP fusion protein was induced by urea in both E. coli DH5a and P. mirabilis HI4320. Thereafter, Western blotting was carried out with antiserum raised against GFP. The results are shown in FIG. 1.

As shown in FIG. 1, the expression level correlated with the urea concentration tested (from 0-500 mM), the highest induction being seen at 200-500 mM urea.

Fluorescent E. coli and P. mirabilis bacteria were observed by fluorescence microscopy following urea induction, and the fluorescence intensity of GFP in cell lysates was measured by spectrophotofluorimetry.

### EXAMPLE 2

### UreR Binds Urea Using a Mechanism Distinct From That in the Urease Active Site

Urease catalyzes the hydrolysis of urea by coordinating the substrate into the catalytic site using specific amino acid residues and two nickel ions. The crystal structure of Klebsiella aerogenes urease (Jabri et al, Science, 268:998-1004 (1995)), which shares 72.5% amino acid sequence identity for its ureC subunit with P. mirabilis urease, predicts that specific residues, Asp360, Cys319, His134, 136, 246, 272, 320 and Lys217 (corresponding residue numbers for P. mirabilis urease) coordinate the nickel ions which are known to be required for urea binding in the catalytic site (Jabri et al, supra). Since urease contains the only characterized urea binding domain, it was hypothesized in the present invention that these four amino acid residues and nickel ions may also be responsible for the binding of urea by UreR.

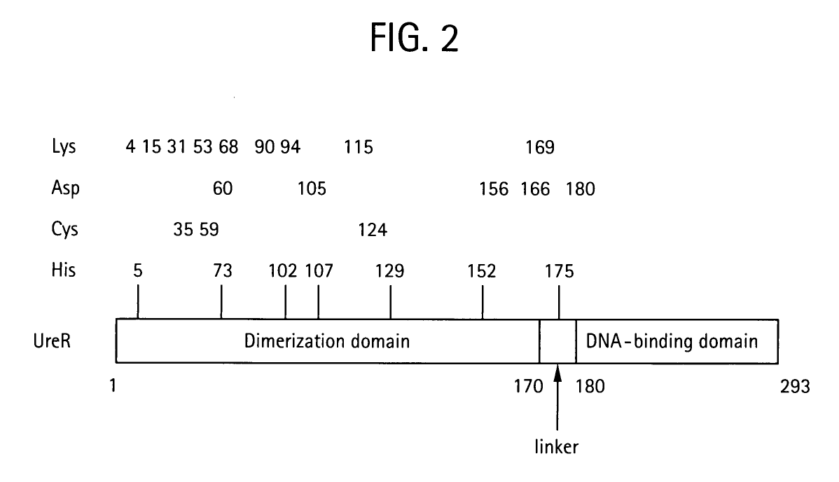

To test this hypothesis, each His, Lys, Asp, and Cys residue in UreR was altered prior to the first helix-turn-helix (residues 186-205), each to Ala, which has a negative ΔG of formation for both α-helices and β-sheets and is abundant in the wild-type protein (see FIG. 2).

More specifically, wild-type ureR was cloned into the NcoI/XhoI site of pBAD/MycHisA (Invitrogen) to create pCP016 (Poore et al, supra). This construct was used to create alanine mutants using overlapping PCR site-directed mutagenesis as described by Ho et al, Gene, 71:51-59 (1989); and Poore et al, supra. Full-length mutant ureR PCR products were then constructed by ligation into the NcoI/XhoI site of pBAD/MycHisA. Following PCR mutation to Ala, all of the cloned products in the pBAD vector were verified by sequencing. The constructs were then cotransformed with the reporter vector P_{ureD}-lacZ into E. coli Top10 (pCC142) (Invitrogen). The β-gal reporter construct pCC042 (tetR) carries a transcriptional fusion of the ureD promoter (pureD) and lacZ (Poore et al, supra). Unless otherwise stated, strains were cultured in Luria broth supplemented with ampicillin (100 μg/ml), kanamycin (50 μg/ml), or tetracycline (15 μg/ml) as required. Thereafter, activity was determined using a β-galactosidase reporter assay in the absence and presence of 100 mM urea.

More specifically, overnight cultures were diluted 1:10 into fresh medium supplemented with ampicillin. Mid-exponential cultures were induced with 0.2% (w/v) arabinose and either no urea or 100 mM urea for 1 h. Bacterial suspensions were placed on ice for 10 min and then permeabilized with 0.1% (w/v) SDS (50 μl) and chloroform (100 μl) at room temperature for 30 min. Aliquots (20-50 μl) were diluted to 1.0 ml in Z buffer comprising 200 μl o-nitrophenyl-β-D-galactopyranoside (4.0 mg/ml) and were incubated at room temperature. Timed reactions were stopped by addition of 1.0 M Na₂CO₃ (500 μl) and the optical densities at 420 nm and 550 nm were used to calculate Miller units for each assay (Platt et al, “Assays of β-galactosidase Activity”, In: Miller (ed.), Experiments in Molecular Genetics, Cold Spring Harbor Press, Cold Spring Harbor, N.Y., pages 352-355 (1972)). In this assay, a significant reduction in activity indicates that one of the four residues is involved in nickel coordination and/or urea binding. The results, which are shown in Table 1 below, are expressed as the average of at least three independent experiments.

TABLE 1
β-galactosidase Reporter Activity
by UreR Alanine Substitution Mutants

β-Galactosidase
Activity (Miller units)

| UreR | 0 mM urea | 100 mM urea |
|---|---|---|
| Wild-type | 632 ± 203 | 3709 ± 1570 |
| pBAD vector | 54 ± 15 | 53 ± 16 |
| H5A | 541 ± 304 | 3387 ± 845 |
| H73A | 410 ± 101 | 3082 ± 291 |
| H102A | 2141 ± 522^{b} | 4229 ± 296 |
| H107A, H186A | 318 ± 53^{a} | 3486 ± 647 |
| H129A | 584 ± 105 | 2610 ± 377 |
| H152A | 313 ± 102^{a} | 2986 ± 377 |
| H175A | 2871 ± 43^{b} | 4770 ± 0262 |
| D60A | 656 ± 234 | 3216 ± 529 |
| D105A | 590 ± 96 | 2971 ± 154 |
| D156A | 714 ± 247 | 3002 ± 218 |
| D166A | 96 ± 25^{a} | 1032 ± 588^{a} |
| D180A | 285 ± 192^{a} | 2045 ± 385 |
| C35A | 477 ± 223 | 2974 ± 246 |
| C59A | 803 ± 250 | 3047 ± 240 |
| C124A | 359 ± 167^{a} | 2045 ± 385 |
| C35A, C124A | 580 ± 230 | 2357 ± 522 |
| K4A | 111 ± 53^{a} | 643 ± 246^{a} |
| K15A | 278 ± 106^{a} | 2666 ± 761 |
| K31A | 391 ± 126 | 2495 ± 633 |
| K53A | 853 ± 498 | 2745 ± 633 |
| K68A | 449 ± 132 | 2834 ± 1041 |
| K90A | 1359 ± 608^{b} | 3361 ± 1731 |
| K94A | 595 ± 323 | 2918 ± 504 |
| K115A | 527 ± 108 | 3025 ± 626 |
| K169A | 112 ± 55^{a} | 1655 ± 514^{a} |

^{a}significantly lower than wild-type (p < 0.05)
^{b}significantly higher than wild-type (<0.05)

As shown in Table 1 above, reporter activity for most mutant UreR constructs did not differ significantly from that measured for the wild-type. It has been previously reported that two His to Ala mutations at positions 102 and 175 resulted in constitutive expression of β-galactosidase in this reporter assay (Poore et al, supra). These results were recently confirmed by Gendlina et al, J. Biol. Chem., 277:37349-37358 (2002). In addition to these two mutants, K90A also displayed significantly increased activity only in the absence of urea. With the exception of these three mutants, site-directed mutation of single amino acid residues did not significantly disrupt activity of UreR (ability to positively activate urease genes) as has been observed for such mutations within the urease catalytic site (loss of urease activity). When compared to the urease enzyme catalytic urea binding site, these data suggest a different type of urea binding motif is utilized by UreR.

### EXAMPLE 3

### Construction of UreR Tryptophan Mutants

It was hypothesized in the present invention that urea binds directly to UreR resulting in a conformational change in the protein that increases the avidity of its interaction with DNA, i.e. UreR-inducible promoters. Local conformational changes in a protein can often be tracked by monitoring changes in the fluorescence of Trp or Tyr residues. To study the UreR conformational effects upon addition of urea, it was necessary to purify recombinant UreR. Initially, tryptophan luminescence was used to investigate the structural changes in protein conformation before and after urea binding. However, there are no Trp residues in the N-terminal half of UreR to monitor by fluorescence spectroscopy. Thus, to demonstrate a direct interaction between UreR and urea, PCR site-directed mutagenesis was used to construct four independent Tyr to Trp point mutations at amino acids 28, 54, 106 and 151 of full-length UreR. In these mutants, a single Trp was introduced into the UreR N-terminus with which to conduct measurements. Mutations at positions 106 and 151 were selected based on their proximity to previously identified UreR dimerization regions. In addition, two additional N-terminal Trp residues at position 28 and 54 were also selected.

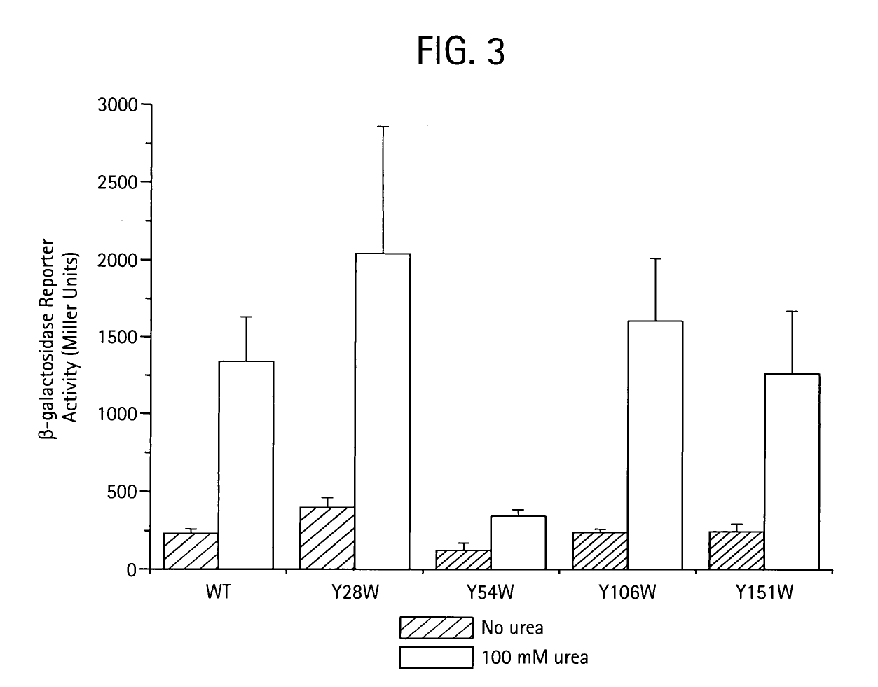

The UreR Trp point mutants were confirmed by sequencing and were tested for the ability to induce β-galactosidase expression using the P_{ureD}-lacZ reporter assay in the presence of 100 mM urea as described above. The results are shown in FIG. 3.

As shown in FIG. 3, UreR point mutants Y28W, Y106W and Y151W displayed activity comparable to that measured for the wild-type protein. While the addition of 100 mM urea produced a 2.8-fold induction in β-galactosidase activity for Y54W UreR, this mutation produced an overall reduction in the level of activation seen compared to wild-type UreR. One explanation among several possibilities (e.g., loss of protein stability) is that Tyr54 resides in a UreR domain that binds urea.

### EXAMPLE 4

### Urea Binds Directly to Purified nUreR

A. Production of UreR Truncated Mutants

To test the hypothesis in the present invention that potential structural changes in the UreR N-terminal region can be detected after urea binding, a 564-bp UreR fragment encoding the first 184 of a total of 293 amino acids of P. mirabilis UreR was cloned into the NcoI/BamHI site of pQE60 (Qiagen) to generate a translational fusion with a C-terminal His-tag.

More specifically, truncated versions of ureR Trp mutants were constructed by PCR amplification of a 564-bp fragment (corresponding to amino acids 1-184) comprising the UreR N-terminal and linker domains (nUreR) using the following oligonucleotides:

(SEQ ID NO:1)

5′-AAAAACCATGGAATACAAACACATACTTTCTTCTAAC-3′, and

(SEQ ID NO:2)

5′-TTTTTTGGATCCTTGCGGATCTTGTGTTATTAGATGAGT-3′.

The 564-bp fragment fragment was cloned into the NcoI/BamHI site of pQE60 to produce a translational fusion between N-terminal UreR (nUreR) with the 6-His tag present in the vector. This cloning strategy resulted in the production of four single Trp proteins of nUreR-6xHis and unmodified nUreR. The truncates, lacking the C-terminal DNA-binding domain, were then expressed and purified.

Expression of the various forms of nUreR was performed based on the method of Strachan et al, J. Appl. Microbiol., 87:410-417 (1999), using E. coli M15 (Qiagen) carrying pREP4 (kan^{R}). For each strain, a single colony was grown overnight in 5.0 ml LB supplemented with ampicillin (100 μg/ml) and kanamycin (50 μg/ml). A 2% inoculum of overnight culture was added to 50 ml of Terrific broth (TB) supplemented with 1.0% (w/v) glucose, ampicillin (100 μg/ml), and kanamycin (50 μg/ml) and the flasks were cultured at 37° C. with aeration (200 rpm) for approximately 8 h. After centrifugation (5000×g, 10 min, 4° C.) of the culture, the cell pellet was resuspended in fresh 50 ml of TB medium and grown overnight at 25° C. with aeration (200 rpm). Cells were harvested by centrifugation and resuspended in 50 ml of TB medium containing ampicillin (100 μg/ml) only. Protein expression was induced by incubation with 1.0 mM IPTG for 3 h at 25° C.

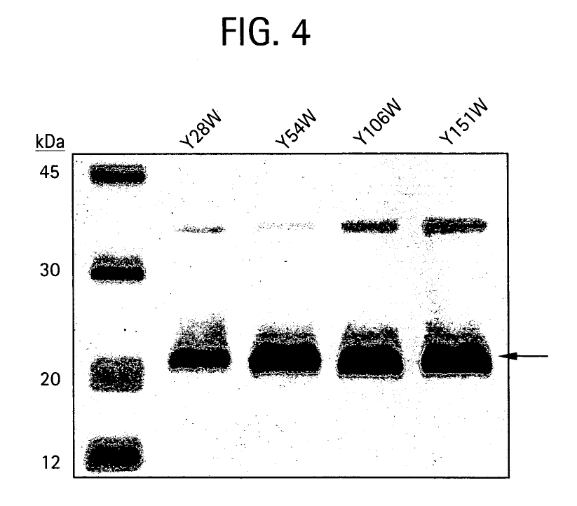

nUreR-His wild-type and mutants were then purified using Ni-NTA resin (Qiagen) basically in accordance with the manufacturer's protocol. All buffers used for nUreR purification contained protease inhibitors and 10 mM β-mercaptoethanol. Bacteria isolated by centrifugation were resuspended in 4.0 ml of binding buffer comprising 50 mM phosphate buffer, pH 8.0, 200 mM NaCl, 5.0 mM imidazole, and were disrupted by two passages through a pre-chilled French Pressure cell at 18,000 psi. Cell debris was removed by centrifugation (20,000×g, 20 min, 4° C.), and the cellular extract was incubated with 1.0 ml of pre-charged Ni-NTA resin (Qiagen, Inc.) for 1 hr at 4° C. before application to a column. After the resin was washed with 10 column volumes of binding buffer containing 55 mM imidazole, the remaining bound protein was eluted with 4 column volumes of binding buffer containing 250 mM imidazole. The eluted protein fractions (0.5 ml) were concentrated using YM-10 Microcon filter (Amicon), and were stored at 4° C., where they were stable for at least 3 weeks. Typical yields from this procedure were 2.5-3.5 mg of total protein. The resulting purified nUreR-His was resolved on a 12% (w/v) SDS-polyacrylamide gel, stained with 0.2% (w/v) Coomassie blue in water to visualize the predicted 35.4 kDa protein band, and excised from the gel. nUreR purity was estimated at ≧95% on the Coomassie blue-stained SDS-polyacrylamide gel (FIG. 4).

To raise antiserum against UreR, the gel fragment was emulsified in Freund's complete adjuvant and subcutaneously injected into New Zealand White rabbits (approximately 100 μg protein/rabbit). Booster injections of UreR-MycHis (50 μg protein/rabbit), emulsified in Freund's incomplete adjuvant, were given 4 and 6 weeks after the initial immunization. Blood, collected 2 weeks after the final booster, was centrifuged to remove erythrocytes and adsorbed with E. coli proteins conjugated to agarose beads (Sigma A-2210) according to manufacturer's protocol. The truncated mutant proteins were observed to positively react with the polyclonal antiserum prepared against the full-length UreR on Western blots.

B. Trp Fluorescence

Trp fluorescence is very sensitive to changes in local microenvironment and has been used extensively to study protein structure, as well as ligand-binding interactions (Eftink, Methods of Biochemical Analysis, 35:127-205 (1991)). It was hypothesized in the present invention that binding of urea by UreR would cause a structural change in the protein that could be detectable by fluorescence spectroscopy. Thus, this approach was employed to analyze the recombinant purified nUreR Trp mutants for their ability to bind to urea.

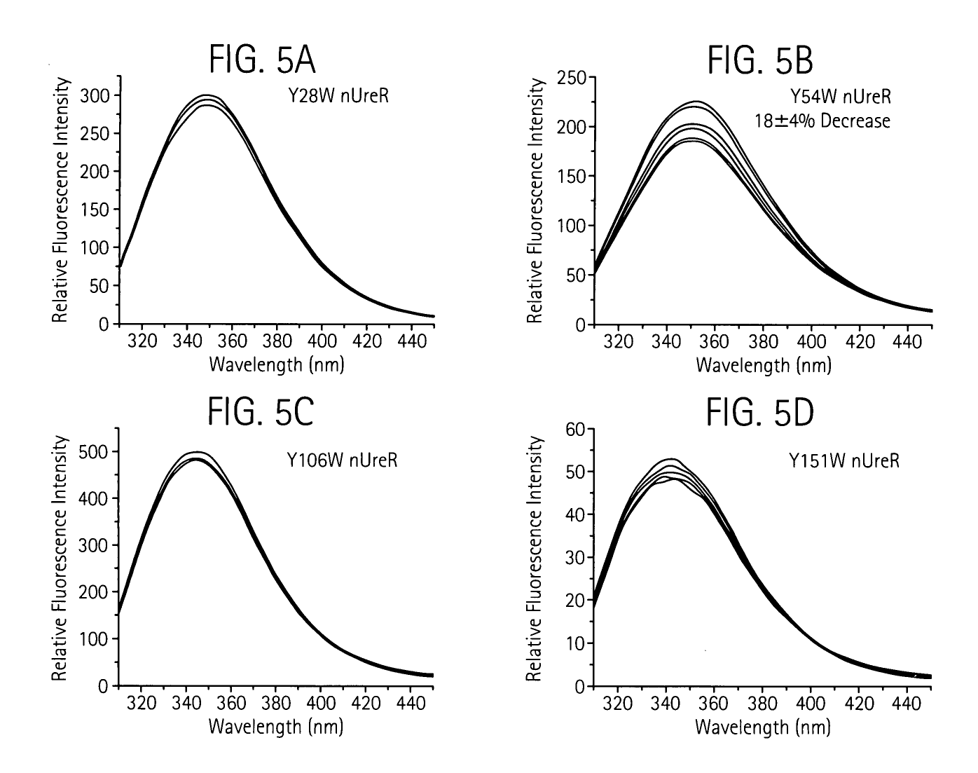

More specifically, Trp emission spectra were collected at room temperature using a Varian Cary Eclipse spectrofluorometer. Purified nUreR Trp mutants (5.0 μM) were excited at 280 nm and urea-dependent emission spectra were measured on the same sample following titration with increasing concentrations (0-150 mM) of urea. The results are shown in FIG. 5.

As shown in FIG. 5, Y54W nUreR diplayed the largest decrease (18%) in Trp emission intensity upon the addition of urea.

Time resolved measurements of the four Trp mutants revealed small, but measurable changes in the lifetime components in the absence and presence of urea.

Identical data were collected at 298 nm suggesting that Trp was responsible for the spectral changes observed without contributing Tyr luminescence.

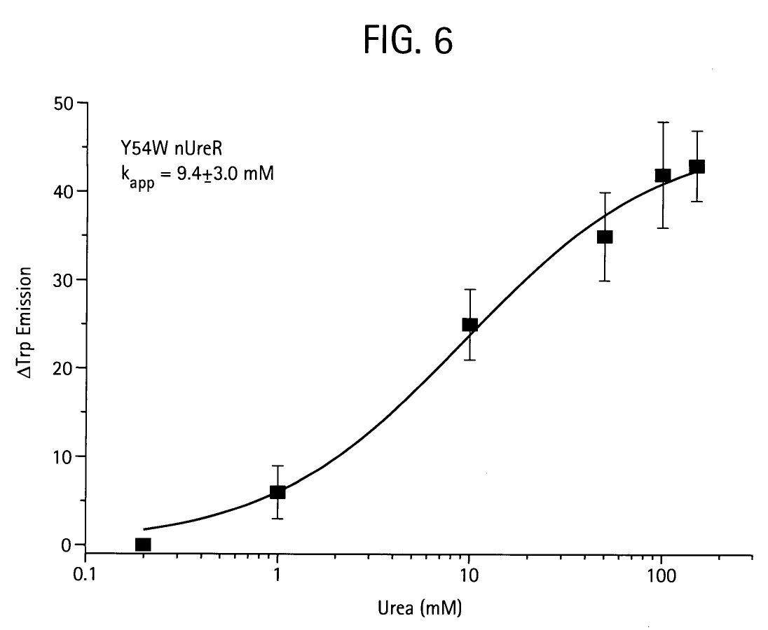

The decrease in Trp fluorescence of Y54W nUreR was used to calculate a urea binding constant for this mutant protein. The corresponding Hill parameters were invoked using the curve fitting functions packaged with the Microcal Origin 6.0 data analysis graphing program (Northampton, Mass.). Using this process, a urea binding constant of 9.0±3.0 mM was calculated for the interaction with nUreR (FIG. 6).

Trp fluorescence emission spectra from 300-450 nm were taken in the presence of increasing concentrations of urea (0, 0.05, 1, 10, 100 and 150 mM). The results are shown in Table 2 below.

TABLE 2
Spectral Properties of nUreR Trp Mutants

| | | | <τ>^{a, b} | |
|---|---|---|---|---|
| UreR Trp | % Decrease in | Emission Max | (ns) | |
| Mutant | Trp Emission | (nm) | No Urea | 150 mM urea |
| Y28W | 3.6 ± 1 | 350 | 5.04 | 4.97 |
| Y54W | 18 ± 4 | 353 | 5.23 | 5.52 |
| Y106W | 3.4 ± 1 | 344 | 5.09 | 4.60 |
| Yl51W | 9 ± 2 | 340 | 1.98 | 1.95 |

^{a}<τ> = αᵢτᵢ
^{b}Error for the phase and modulation were taken to be: δφ = 0.4 and δm = 0.007

As shown in Table 2 above, the emission maximum varied from 342-353 nm indicating that the fluorescent Trp residue was in a slightly different microenvironment for each mutant.

While nUreR mutants Y28W, Y106W, and Y151W displayed subtle changes in Trp emission, Y54W nUreR demonstrated a significant and saturatable decrease (18±4%) in Trp emission in response to increasing urea concentrations. This indicates that the local environment around amino acid residue 54 changes when the protein is exposed to urea.

To fully characterize the mutant proteins, the Trp fluorescence lifetime for each mutant was determined in the absence or presence of 100 mM urea.

Trp fluorescence lifetime determinations were made using the frequency-doubled output (298 nm) of pyridine dye laser pumped with a mode-locked Ar+ laser, available at the Center for Fluorescence Spectroscopy at UMB as described by Beechem et al, “The Global Analysis of Fluorescence Intensity and Anisotropy Decay Data: Second-Generation Theory and Programs”, In J. R. Lakowicz (ed.), Topics in Fluorescence Spectroscopy, Vol. 2, Plenum Press, New York, pages 241-305. (1991); and Lakowicz et al, “Frequency-Domain Fluorescence Spectroscopy”, In J. R. Lakowicz (ed.), Topics in Fluorescence Spectroscopy, Vol. 1, Plenum Press, New York, pages 293-355 (1991).

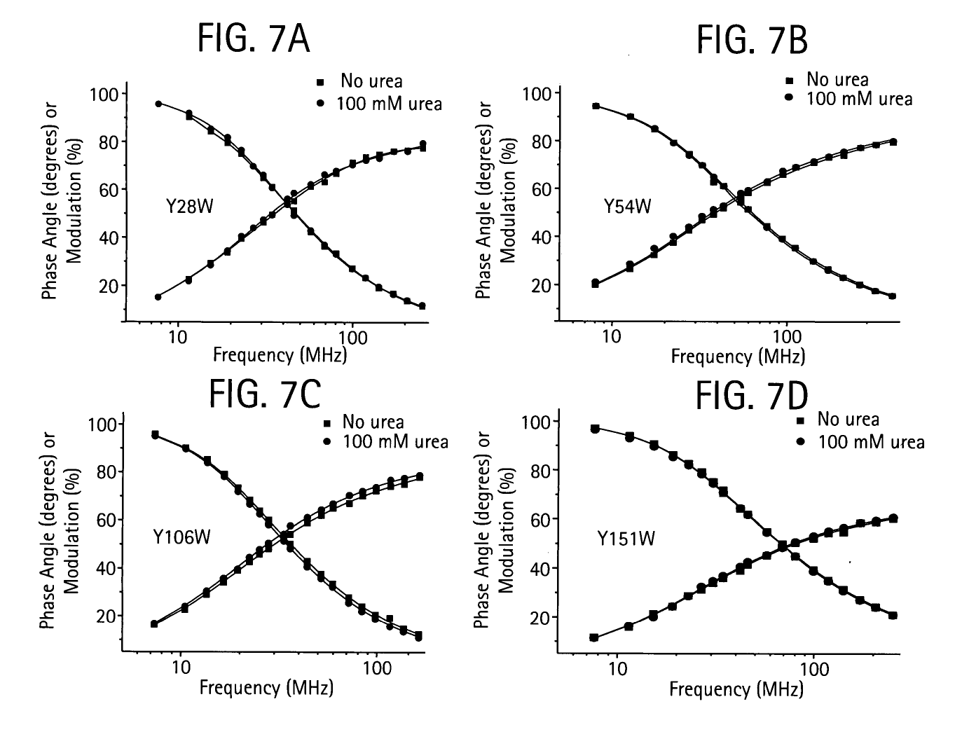

More specifically, frequency-domain lifetime data for single Trp nUreR mutants (2.0 μM) with and without 100 mM urea were performed using a GHz instrument (Laczko et al, Rev. Sci. Instr., 61:9231-9237 (1990)). Excitation of Trp was accomplished using the frequency-doubled output of a rhodamine-6-G dye laser (295 nm) synchronously pumped by a mode-locked argon ion laser (514 nm). Measurements were made with polarizers set to magic angle conditions (0° and 54° for excitation and emission polarizers, respectively). DCS (4′dimethylamino)-4′-cyanostilbene) in methanol with a lifetime of 0.46 nm was used as reference. Analysis of the data was performed using nonlinear least-squares as previously described by Lakowicz et al, Biophys. J., 46:463-77 (1984). The results are shown in FIGS. 7A-7D.

As shown in FIGS. 7A-7D, nUreR mutants Y28W, Y54W and Y106W displayed lifetimes of approximately 5 ns, while Y151W exhibited a much lower value of 2 ns. However, the lifetime values did not significantly change in the presence of 100 mM urea for any Trp mutant.

### EXAMPLE 5

### Covalent Labeling of a Cys59 nUreR Mutant With NBD

To confirm the importance of the N-terminal region of nUreR to urea binding, Cys59 was labeled because of its proximity to the urea-responsive Trp54. To accomplish this Cys35 and Cys124 were site-specifically mutated to Ala, leaving Cys59 as the sole Cys residue in nUreR. The full length 293-amino acid protein with these two mutations retained urea inducibility of urease reporter construct (Table 1 above). The activity of the double Cys mutant in this assay was not significantly different from the unaltered UreR (Table 1 above).

The sequences encoding the nUreR polypeptides were cloned and the protein was purified using the 6×-His tag as described above. The sole remaining Cys residue (Cys59) in nUreR was covalently modified with NBD amide-iodoacetamide as described by the manufacture Molecular Probes, Eugene, Oreg. Specifically, the components were incubated for 2 hrs in the dark in 50 mM Tris-HCl (pH 7.2) at room temperature. The reaction was quenched by the addition of β-mercaptoethanol, and the conjugated protein was separated from the reagents by dialysis or gel filtration chromatography. The NBD-labeled nUreR was excited at 488 nm and fluorescence at 535 nm was measured in the absence and presence of 1.0 mM urea. The results are shown in FIG. 8.

As shown in FIG. 8, a significant increase in NBD fluorescence emission was observed in the presence of 1.0 mM urea in quadruplicate experiments.

### Discussion

The model for UreR activity suggested that UreR binds urea resulting in a conformational change that increases the affinity for the ureD and ureR promoter regions to activate transcription by RNA polymerase. In the present invention, a direct interaction between recombinant UreR and urea has been demonstrated for the first time. The data herein suggests that the UreR urea binding domain is located in the N-terminal half of the polypeptide in the proximity of amino acids 54 to 59.

For the vast majority of AraC family members such as UreR, ligand binding ability is found in the N-terminal domain (Gallegos et al, Microbiol. Mol. Biol. Rev., 61:393-410 (1997)). It was hypothesized in the present invention that UreR would conform to this general principle. Site-directed mutagenesis was used to attempt to identify the specific amino acid residues responsible for the UreR interaction with urea. For the most part, individual ureR mutation of all N-terminal Asp, Cys, His, or Lys to Ala did not diminish the ability of mutant UreR to activate transcription from the ureD promoter following the addition of urea (Table 1 above). Three of the mutants resulted in constitutively induced urease genes in the absence of urea. Individual Ala substitutions at His102, His175 and Lys90 induced ureD-lacZ reporter in the absence of urea to a level that is not significantly different (p<0.001) from native UreR induced with 100 mM urea. While these mutations may have caused an allosteric shift in UreR resulting in avid binding to UreR-inducible promoters, they do not appear to be directly involved in binding urea. Since these amino acids are known to be important for urea binding within the catalytic site of urease, these data suggest a new type of urea binding motif is present in UreR.

To test for the direct interaction between urea and UreR, four functionally active UreR Trp mutants were prepared to monitor any urea-induced changes in the fluorescence emission properties. While there are no Trp residues in the UreR N-terminal domain, there are seven Tyr residues distributed throughout the first 181 amino acids of UreR. Site-directed mutagenesis of plasmid pCP016 (on which ureR is carried) was used to create four single Trp mutants in which the Tyr residues located at amino acid numbers 28, 54, 106 and 151 were individually replaced with Trp. The choice of Tyr residues was guided by both a theoretical UreR model, as well as experimental evidence suggesting that these regions play essential roles in UreR activity. Expression of each mutant was verified in arabinose-induced E. coli Top10 crude lysates by performing Western blot analysis with anti-UreR antiserum; no evidence of degradation was noted. Additionally, the ability of each mutant to activate the ureD-lacZ reporter system was confirmed (FIG. 3). The mutant nucleic acid sequences were PCR amplified and cloned into pQE60 to create a translational fusion between nUreR and a 6xHis affinity tag. The nUreR-6xHis single Trp mutants were individually expressed in an E. coli M15 (pREP4) background and purified using Ni-NTA affinity chromatography (FIG. 4).

The Trp emission spectrum of each nUreR mutant in the presence and absence of urea revealed in one mutant a substantial change of environment of the Trp residue. Mutants Y28W, Y106W and Y151W displayed small or insignificant changes in the fluorescence properties of Trp (FIG. 5). In contrast, Y54W nUreR displayed an 18% decrease in Trp emission intensity upon the addition of increasing urea concentrations suggesting that the urea binding domain may be localized to this region of the protein. From these data, a urea binding constant of 9.0±3.0 mM for Y54W nUreR was obtained (FIG. 6). Calculation of the theoretical binding constant may be complicated by the Tyr54Trp amino acid substitution in the very region that is hypothesize to represent the urea binding domain.

While the Y54W nUreR mutant gave satisfactory results, site-directed mutagenesis was used to create a nUreR mutant with a single Cys residue at position 59 that was covalently modified with the environmentally sensitive fluorophore NBD iodoacetamide. A large increase in fluorescence intensity was observed upon incubation with 1.0 mM urea (FIG. 8). These dramatic changes in fluorescence confirm the importance of this specific region of UreR to urea binding and indicates that nUreR can serve as the basis for a new biosensor in the range of physiological concentrations of urea.

Previously, using confocal microscopy and urea-inducible green fluorescent protein (GFP) fusions with ureD expressed from a plasmid in P. mirabilis HI4320, full induction of expression from the ureD promoter was observed (Zhao et al, Infect. Immun., 66:330-335 (1998)). Since the level of urea in the kidneys approaches 500 mM and the calculated binding constant is in the 10 mM range, UreR is predicted to be saturated, and thus fully activated. While this value seems reasonable for the physiological expression of urease during the host infection, the wild-type binding constant may be lower since a reduction in the inducibility for this mutant was observed in a functional reporter assay (FIG. 3). Indeed, Gendlina et al, supra recently reported a urea binding constant of 0.2 mM for the Providencia stuartii plasmid-encoded UreR (67% amino acid sequence identity with P. mirabilis UreR). Their work takes advantage of the endogenous Trp residue at position 186, which is located at the beginning of the first helix-turn-helix DNA binding motif. While using this site for determination of a binding constant was acceptable, this residue location was not ideal for localizing the urea binding site within the N-terminal domain.

While the UreR three-dimensional architecture is likely important for proper urea binding, the data herein indicates that the region containing amino acids 54 to 59 may be in close proximity to the UreR urea binding site. A role for nickel in the UreR urea binding site similar to that found in the active site of urease has been suggested. Given the large pool of alanine mutants developed here (Table 1 above), it is now believed that it is unlikely that nickel is involved in urea binding by UreR.

While the invention has been described in detail and with reference to specific embodiments thereof, it will be apparent to one skilled in the art that various changes and modifications can be made therein without departing from the spirit and scope thereof.

What is claimed:

1. A method for assaying for the presence of urea in a test sample comprising:
(A) contacting said test sample with a member selected from the group consisting of:
(1) a polypeptide comprising at least a urea binding fragment of UreR, wherein a tryptophan residue has been substituted for at least one amino acid residue in said fragment,
(2) a polypeptide comprising at least a urea binding fragment of UreR covalently linked to a solvent-sensitive fluorophore,
(3) a polypeptide comprising at least a urea binding fragment of UreR in the presence of a fluorescent thiourea, and
(4) a fusion protein comprising a polypeptide at least a urea binding fragment of UreR and a fluorescent protein;
(B) measuring
(1) a change in intrinsic fluorescence of tryptophan in said polypeptide,
(2) a change in fluorescence of said solvent-sensitive fluorophore,
(3) a change in fluorescence of said fluorescent thiourea, or
(4) a change in fluorescence of said fluorescent protein, respectively,
as a result of binding of urea present in said test sample to said polypeptide.

2. The method of claim 1, wherein said polypeptide comprises amino acids 1-184 of UreR.

3. The method of claim 1, wherein said UreR is selected from the group consisting of Proteus mirabilis UreR, Providencia stuartii UreR, E. coli UreR, Corynebacterium glutamicum UreR and Vibrio parahaemolyticus UreR.

4. The method of claim 1, wherein said UreR is Proteus mirabilis UreR amino acids 1-184 which has been mutated so as to substitute tyrosine at position 54 with tryptophan.

5. The method of claim 1, wherein said solvent-sensitive fluorophore is selected from the group consisting of:
4-chloro-7-nitrobenz-2-oxa-1,3-diazole,
5-((((2-iodoacetyl)amino)ethyl)amino) naphthalene-1-sulfonic acid,
7-fluorobenz-2-oxa-1,3-diazole-4-sulfonamide,
2-(4′-maleimidylanilino)naphthalene-6-sulfonic acid, sodium salt, and
1-(2-maleimidylethyl)-4-(5-(4-methoxyphenyl)oxazol-2-yl) pyridinium methanesulfonate,

6. The method of claim 1, wherein said polypeptide is Proteus mirabilis nUreR wherein the sulfhydryl of the cysteine at position 59 of UreR is covalently linked to said solvent-sensitive fluorophore, and wherein said solvent-sensitive fluorophore is NBD iodacetamide.

7. The method of claim 1, wherein said fluorescent thiourea is selected from the group consisting of fluorescein thiourea, tetramethylrhodamine thiourea and Eosin thiourea.

8. The method of claim 1, wherein said fluorescent protein is selected from the group consisting of Aequorea Green Fluorescent protein, Yellow Fluorescent Protein, Blue Fluorescent Protein, and DsRed.

9. The method of claim 1, wherein said change in fluorescence is at least one member selected from the group consisting of a change in fluorescence intensity, a change in fluorescence anisotropy, a change in fluorescence polarization, a change in fluorescence emission and a change in excitation spectrum.

10. The method of claim 7, wherein said change in fluorescence is measured using an optical fiber.

11. A method for assaying for urea in a test sample comprising:
(A) contacting a test sample with a polypeptide comprising a UreR, and a fluorescent-labeled oligonucleotide comprising at least a UreR binding fragment of a UreR-inducible promoter, wherein said contacting is carried under conditions such that said UreR binds to said oligonucleotide; and
(B) measuring a change in anisotropy as a result of binding of urea present in said test sample to a complex formed between said oligonucleotide and said polypeptide.

12. The method of claim 11, wherein said polypeptide comprises amino acids 1-184 of UreR.

13. A kit for assaying for the presence of urea in a test sample comprising:
(A) a member selected from the group consisting of:
(1) a polypeptide comprising at least a urea binding fragment of UreR, wherein a tryptophan residue has been substituted for at least one amino acid residue in said fragment,
(2) a polypeptide comprising at least a urea binding fragment of UreR covalently linked to a solvent sensitive fluorophore,
(3) a polypeptide comprising at least a urea binding fragment of UreR in the presence of a fluorescent thiourea, and
(4) a fusion protein comprising a polypeptide comprising at least a urea binding fragment of UreR and a fluorescent protein; and
(B) a member selected from the group consisting of a buffer solution, a sample diluent, a vessel for mixing said polypeptide and buffer solution or sample diluent, and a standard.

14. The kit of claim 13, wherein said polypeptide comprises amino acids 1-184 of UreR.

15. An apparatus for detecting urea, comprising:
a first chamber;
a second chamber; and
a urea-permeable membrane between the first chamber and the second chamber, the urea-permeable membrane configured to allow urea to pass between the first chamber and the second chamber.

16. The apparatus according to claim 15, wherein the second chamber comprises a first transparent window for the transmission of an excitation light and a second transparent window for observing fluorescent anistropy within the second chamber.

17. The apparatus according to claim 15, wherein the first chamber further comprises a urea containing aqueous sample, and the second chamber further comprises a UreR and a fluorescent-labeled oligonucleotide comprising at least a UreR binding fragment of a UreR-inducible promoter.

18. The apparatus according to claim 15, wherein the a urea-permeable membrane is a dialysis membrane.

19. A system for detecting urea, comprising:
a first chamber;
a second chamber comprising a first transparent window for the transmission of an excitation light and a second transparent window for observing fluorescent anistropy within the second chamber;
a urea-permeable membrane between the first chamber and the second chamber, the urea-permeable membrane configured to allow urea to pass between the first chamber and the second chamber; and
an excitation light source configured to direct an excitation light through the first transparent window.

20. A method for assaying for the presence of urea in an aqueous sample comprising:
providing:
a first chamber having the aqueous sample;
a second chamber having a UreR and a fluorescent-labeled oligonucleotide comprising at least a UreR binding fragment of a UreR-inducible promoter; and
a urea-permeable membrane between the first chamber and the second chamber, the urea-permeable membrane configured to allow urea to pass between the first chamber and the second chamber;
introducing an excitation light into the second chamber; and
observing the presence of fluorescence anistropy in the second chamber.
